SEQUENCE LISTING (2 sequences)

SEQ ID NO: 1 - DNA, 6 bp, Homo sapiens
ggaaga

SEQ ID NO: 2 - RNA, 23 nt, Homo sapiens
uggaagacuagugauuuuguugu

(19) United States
(12) Patent Application Publication
(10) Pub. No.: US 2017/0088836 A1
(43) Pub. Date: Mar. 30, 2017

(54) TREATMENT AND PREVENTION OF ADRENOCORTICAL CARCINOMA

(71) Applicants: Anthony Glover, New South Wales (AU); Jing Ting Zhao, New South Wales (AU); Bruce Robinson, New South Wales (AU); Stanley Sidhu, New South Wales (AU)

(72) Inventors: Anthony Glover, New South Wales (AU); Jing Ting Zhao, New South Wales (AU); Bruce Robinson, New South Wales (AU); Stanley Sidhu, New South Wales (AU)

(21) Appl. No.: 15/274,961

(22) Filed: Sep. 23, 2016

Related U.S. Application Data
(60) Provisional application No. 62/232,672, filed on Sep. 25, 2015.

(30) Foreign Application Priority Data
Sep. 25, 2015 (AU) 2015230812

(51) Int. Cl. C12N 15/113

(52) U.S. Cl. CPC C12N 15/113; C12N 2310/141

(57) ABSTRACT

The invention relates to compositions and methods utilising miR-7 microRNA

### STATEMENT REGARDING ELECTRONIC FILING OF A SEQUENCE LISTING

A Sequence Listing in ASCII text format, submitted under 37 C.F.R. §1.821, entitled 9975-8 ST25.txt, 648 bytes in size, generated on Sep. 23, 2016 and filed via EFS-Web, is provided in lieu of a paper copy. This Sequence Listing is hereby incorporated by reference into the specification for its disclosures.

### FIELD OF THE INVENTION

The invention relates to treatments for adrenocortical carcinoma.

### BACKGROUND OF THE INVENTION

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

Adrenocortical carcinoma (ACC) is a rare but aggressive cancer. ACC has a poor prognosis, with limited treatment options and an overall 5-year survival of less than 35% for metastatic disease [1].

Given the rarity of the disease, it has been difficult to unravel the pathogenesis of ACC, particularly at a molecular level. As a result, the current therapeutic options for ACC are limited, with medical and radiation therapy remaining the main approach, complementary to surgery. For advanced or metastatic ACC, the current standard chemotherapy regimen is a combination of drugs including doxorubicin, cisplatin, etoposide and mitotane. The most recent clinical trial comparing this combination to streptozotocin and mitotane (FIRM-ACT study [2]) highlighted the poor outcomes for ACC patients, where the conventional treatment was found to have a response rate of only 23%. For patients whose tumours progress despite standard chemotherapy, there are currently no proven second-line options.

In an effort to better understand the molecular pathogenesis of ACC, a number of molecular markers have been identified as prognostic markers, as well as therapeutic targets. For example, Ki67 (an indicator of the proliferative activity of a tumour) has both been used to predict recurrence of ACC or prognose the severity of the disease [3]. More recently, microarrays have been used to identify microRNAs as predictors of poor prognosis in ACC. The miRNAs miR-195 and miR-483-5p were identified as being dysregulated in ACC compared with adrenocortical adenoma (ACA)[4].

miRNAs are non-coding, 20-24 nucleotide RNA molecules that regulate gene expression in a sequence-specific manner. miRNAs are thought to have a wide range of roles in development, differentiation, growth and apoptosis. miRNAs interact with target nucleic acid transcripts (mRNAs) containing complementary sequences, and may induce cleavage of the mRNA or inhibit translation. miRNA complexes also use other mechanisms to block protein expression [5] and induce both direct and indirect transcriptional changes [6]. In animals, miRNAs are transcribed from intergenic or intronic DNA as large precursor molecules, terms pri-miRNAs which then are subjected to enzymatic processing by the complexes termed Drosha, Pasha and Dicer, into mature, double-stranded RNA molecules of approximately 22 nucleotides. This duplex, termed the miRNA duplex is incorporated into the RISC complex and the mature miRNA strand is preferentially retained, while the complementary strand (often termed the passenger strand) is discarded.

Although there is increasing recognition that miRNAs are involved in carcinogenesis, it is also known that some miRNAs may act as tumour suppressors, with others act as oncogenes. To further complicate matters, some miRNAs are known to act as oncogenes in one cell type, but as tumour-suppressors in another cell type. Furthermore, although some miRNAs may have altered levels of expression in cancer, it is not always clear whether this is indicative of a role in the pathogenesis of the cancer, or simply an association with the altered physiological state of the cells. As such, one challenge for the use of miRNA in cancer diagnosis, prognosis or therapy is to establish the mechanism of action of any particular miRNA and its role in the pathogenesis of the cancer.

Despite some small advances in the identification of biomarkers of ACC, there remains a significant, unmet clinical need for the treatment of ACC due to its late diagnosis, high rates of recurrence/metastasis and poor response to conventional treatment. There is a need for effective and safe therapies for ACC.

### SUMMARY OF THE INVENTION

The present invention relates to a method of treating adrenocortical carcinoma (ACC) in an individual, the method including providing a therapeutically effective amount of a miR-7 microRNA in the individual, thereby treating ACC in the individual.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: miR-7 is under-expressed in ACC clinical samples. miR-7 expression was under-expressed in clinical samples of ACC compared to normal adrenal cortex (NAC), reduced miR-7 expression was found in the ACC cell lines H295R and SW-13 (3 replicates shown), RNU48 reference gene, median expression shown, data presented as Tukey Box Plot, median expression is represented by the solid line within the box shown, and true outliers (>1.5× interquartile range) are represented by the dots outside the boxes, **** indicates P<0.0001.

FIG. 2: miR-7 inhibits cell proliferation and induces cell cycle arrest. A, B: Expression levels of miR-7 in cell lines H295R and SW13 three and five days after transfection with miR-7 mimic RNU48 reference gene, error bars show SEM, **** indicates P<0.0001. C, D: H295R and SW-13 cell proliferation was reduced following miR-7 replacement compared to miR-NC. Cell proliferation was assessed using MTS assays by three experiments, error bars show SEM, **** indicates P<0.0001. E,F: Following miR-7 replacement, G1 phase of cell cycle was increased by a mean 7.6% in H295R cells and increased by a mean 9.2% in SW-13 cells in miR-7 treated cells compared to miR-NC treated cells. S phase was reduced by a mean 6.9% in H295R cells and reduced by a mean 6.7% in SW-13 cells in miR-7 treated cells compared to miR-NC treated cells. Cell cycle was assessed using flow cytometry with PI staining by three experiments. ** indicates P<0.01, **** indicates P<0.0001, error bars show SEM.

FIG. 3: RAF1 and MTOR are reduced following miR-7 replacement in ACC cell lines. A: Following miR-7 replacement in H295R cells, mRNA levels of RAF1 and EGFR were reduced. GAPDH reference gene, error bars show SEM, ** indicates P<0.01, *** indicates P<0.001. B: Following miR-7 replacement in SW-13 cells, reduced mRNA levels of RAF1, EGFR, EIF4E and MTOR were detected. GAPDH reference gene, error bars show SEM, * indicates P<0.05. C: Following miR-7 replacement in H295R cells, reduced mean protein expression of EGFR, RAF1 and MTOR was detected compared to miR-NC treated cells. Representative images of one experiment shown, number refers to mean densitometry measurement taken from three experiments. * indicates P<0.05. D: Co-transfection of the luciferase-reporter vector containing 3′ UTR of EGFR and RAF1, respectively along with miR-7 mimics suppressed luciferase activity. Assessed by three experiments, mean luciferase activity shown and adjusted to miR-NC activity=1 for both vectors, error bars show SEM, **** indicates P<0.0001.

FIG. 4: Targeted miR-7 replacement using EDV nanoparticles reduces ACC cell line and patient derived xenograft growth. A: EGFR is expressed in ACC, image shows H&E (left) and EGFR (right) stained sections of ACC in patient sample showing diffuse strong EGFR positivity across the tumor, 20× magnification in top images & 600× magnification in bottom images. B: H295R xenografts in (nu/nu) mice were treated with systemic ^{EGFR}EDV™ nanocells containing either miR-7 or miR-NC (n=6 for each group). Arrows indicate days of treatment, mean volumes shown for each group, error bars show SEM, * indicates P<0.05, ** indicates P<0.01. C, D: Repeat experiment of H295R xenografts in (nu/nu) mice treated with four doses and six doses of systemic ^{EGFR}EDV™ nanocells respectively containing either miR-7 or miR-NC (n=6 for each group), mean volumes shown for each group, error bars represent SEM, * indicates P<0.05. E: Primary ACC xenografts were established in (nu/nu) mice and treated with ^{EGFR}EDV™ nanocells containing either miR-7 or miR-NC (n=5 for each group). Arrows indicate days of treatment, mean volumes shown for each group, error bars show SEM, * indicates P<0.05, ** indicates P<0.01

FIG. 5: Systemic miR-7 therapy in vivo leads to inhibition of RAF1, MTOR and CDK1 without evidence of off-target effects. A, B, C, D: Following miR-7 replacement, increased levels of miR-7 were found in H295R xenografts (n=6 for each group) while no significant difference was seen in organs, RNU48 reference gene for xenografts and mouse U6 snRNA reference gene for mouse organs, error bars show SEM, * indicates, P<0.05, NS indicates P-value not significant. E, F, G, H: Following six doses of miR-7 replacement in H295R xenografts, reduced mRNA levels of RAF1, MTOR and CDK-1 were detected, no significant difference in EGFR expression was detected, no significant difference of CDK1, RAF1 or MTOR were detected in mouse liver, lung or kidneys compared to the miR-NC treated xenografts, GAPDH reference gene for xenografts, mouse B2m reference gene for mouse organs, error bars show SEM, ** indicates P<0.01, * indicates P<0.05, NS indicates P-value not significant. I, J, K, L: H&E staining shown for miR-7 treated mice showed no difference in the xenograft, liver, lung or kidney compared to the miR-NC, H&E staining shown for miR-7 treated mice, 200× magnification.

FIG. 6: Reduced protein expression of RAF1, MTOR and CDK1 in mouse xenografts following miR-7 therapy. Following six doses of miR-7 replacement in H295R xenografts, reduced mean protein expression of RAF1, MTOR and CDK1 was detected compared to miR-NC treated xenografts. Representative images of two xenografts per group shown, number refers to mean densitometry measurement taken from five xenograft samples. * indicates P<0.05, NS indicates P value not significant.

FIG. 7: In ACC patient samples, miR-7 expression is inversely associated with CDK1 expression. Median miR-7 expression was higher with low CDK1 expression compared to high CDK1 expression in ACC clinical samples (n=15), high and low CDK1 expression was defined by a sample splitting method by median CDK1 expression of the clinical samples, data presented as Tukey Box Plot, median expression is represented by the solid line within the box shown, there were no true outliers (>1.5× interquartile range), * indicates P<0.05.

### DETAILED DESCRIPTION OF THE EMBODIMENTS

miR-7 Molecules and Sequences

MicroRNAs (miRNAs) are small, non-coding RNA molecules which function as regulatory molecules in plants and animals to control gene expression by binding to complementary nucleic acid sequences on message strands (mRNA). miRNAs can bind to the 3′ untranslated region (3′ UTR) of target mRNAs. In some examples, a “seed” region of approximately 6 to 7 nucleotides near the 5′ end of the miRNA is important to ensure binding to appropriate targets.

miR-7 is an evolutionary conserved miRNA, encoded at three different genomic locations in humans and mice. Without wishing to be bound by theory, the inventors believe that miR-7 is transcribed from each of these three genomic locations into primary miR-7 transcripts, known as pri-miR-7-1, pri-miR-7-2 and pri-miR-7-3. Each of these transcripts is processed in the nucleus by RNAse III endonuclease to form stein-loop precursors, also known as hairpin precursor molecules, and termed pre-miR-7-1, pre-miR-7-2 and pre-miR-7-3. The hairpin precursor molecules are transported to the cytoplasm where they are further processed into short RNA duplexes by the Argonaute protein complex. These duplexes are typically between 21-23 basepairs, and comprise a sense strand which is the mature, functional (or active) miRNA strand, and the complementary (anti-sense) strand, which is sometimes referred to as the passenger strand. When the RNA duplex molecule is taken up by the RISC complex, the passenger strand is ejected and the mature miRNA strand is use to guide binding of the RISC complex to the target sequence on the target mRNA.

All three miR-7 genes give rise to the same mature miR-7 sequence which comprises a “seed region” corresponding to the nucleotide sequence 5′ GGA AGA 3′ (SEQ ID NO:1) which may be important for the binding of the miR-7 mature sequence to its target mRNA. The sequence of mature miR-7, miR-7-5p, is provided in SEQ ID NO:2.

Treatment of Adrenocortical Carcinoma by miR-7 Replacement Therapy

The present invention is based on the finding by the inventors that miR-7 microRNA is significantly under-expressed in adrenocortical carcinomas as compared with adrenocortical adenomas and normal adrenal cortex. Importantly, the inventors have demonstrated that providing miR-7 in adrenocortical carcinoma cells, such that the amount of miR-7 in the carcinoma cell resembles that characteristic of a non-cancerous cell, causes arrest of the cell cycle and prevents proliferation of the tumour cells. As such, the present invention contemplates the use of miR-7 molecules in treating and preventing the progression of adrenocortical carcinomas.

Accordingly, in a first embodiment, the present invention relates to a method of treating adrenocortical carcinoma (ACC) in an individual, the method including providing in the individual an effective amount of a miR-7 microRNA.

The term “miR-7 molecule” as used herein, includes fragments and precursors of a miR-7 microRNA molecule, provided that the molecule comprises a functional miR-7 sequence which, when incorporated into the RISC complex, enables binding of the complex to a miR-7 target mRNA and thereby “mimics” the action of the mature miR-7-5p sequence. The miR-7 microRNA molecule can therefore be any RNA molecule of any length, which can form a double-stranded hairpin structure which is recognised by a cell's RNAi machinery. Upon processing of the double-stranded RNA molecule by Dicer into a double-stranded RNA duplex (of approximately 22 nucleotides in length), the molecule includes an active strand which includes the nucleotide sequence of SEQ ID NO:1. Preferably, it includes the nucleotide sequence of SEQ NO: 2.

In some embodiments, the miR-7 microRNA molecule is an RNA molecule consisting of an active strand having a nucleotide sequence that is at least 80% identical to the sequence of SEQ ID NO: 2. In one embodiment, the RNA molecule is an RNA duplex such that the complementary (or passenger) strand of the duplex comprises a sequence which is at least 60% identical to the sequence which is complementary to SEQ ID NO: 2.

The individual who is being treated in accordance with the methods of the present invention is an individual who has been diagnosed or is suspected of having ACC. Diagnosis of ACC is accomplished by any of the standard methods known in the art and will be familiar to the skilled person. The most common presentation of ACC is due to symptoms of adrenal steroid hormonal excess, with functional tumours making up to approximately 60-75% of ACC cases [7]. An adrenal mass which co-secretes steroids and androgens is highly suggestive to be an ACC [8]. Patients with non-functional tumours usually present with symptoms related to local mass effect such as abdominal discomfort or back pain. However an increasingly common mode of diagnosis is by an incidental finding on modern imaging in the form of an adrenal ‘incidentaloma’.

Diagnosis of ACC typically occurs following surgery to remove an adrenocortical mass. Determination of whether the mass is benign (an adenoma) or malignant (ACC) is by histopathology and the use of the modified Weiss scoring system [9]. The skilled person will be familiar with other means for diagnosis of ACC including use of the Ki67 index.

As used herein, treating, or treatment refers to amelioration of the symptoms associated with a disease or condition. In particular, the disease or condition being treated is a cancer, and in a preferred embodiment, the cancer is adrenocortical carcinoma. As discussed above, it is common for ACC to recur in individuals even after initial treatment whereby there are no detectable signs of ACC in the period immediately after treatment. Accordingly the present invention also contemplates the treatment of a cancer recurrence. In yet a further embodiment, the present invention relates to preventing or delaying the onset of disease symptoms and/or lessening the severity or frequency of the symptoms of the cancer.

In addition to providing a first-line treatment for ACC, the present invention also contemplates the use of miR-7 as an adjuvant treatment for ACC. An adjuvant therapy is a therapy which occurs after a first-line therapy and which may also be used to prevent recurrence of a disease or condition. For example, an individual diagnosed with ACC and who has received a treatment for ACC (such as surgery, chemotherapy or a combination of treatments) may be identified as being at high risk of ACC recurrence and therefore suitable for adjuvant therapy in the form of miR-7 miRNA therapy. Risk of recurrence of ACC can be determined by any known method in the art, including, for example, the use of the Ki67 index [3].

In circumstances where an individual is assessed as being at risk of recurrence of ACC, adjuvant miR-7 therapy may be contemplated in accordance with the method of the present invention. Accordingly, the present invention also relates to a method of preventing the recurrence of ACC in an individual, the method including providing a therapeutically effective amount of a miR-7 microRNA in the individual, thereby preventing the recurrence of ACC in the individual.

As used herein, the term “individual” or “subject” includes animals, such as mammals, including but not limited to primates, livestock and other veterinary species including companion animals. In a preferred embodiment, the individual is a human.

The present inventors have found that introduction of miR-7 microRNA in ACC cells inhibits cell proliferation and cell cycle progression. Accordingly, in a further embodiment, the invention relates to a method of preventing the proliferation and/or cell cycle progression of an adrenocortical carcinoma cell including providing in the cell, an effective amount of a miR-7 microRNA.

Form of the miR-7 microRNAs

The present invention contemplates the use of miR-7 microRNA precursors or derivatives thereof in the methods of treating ACC described herein. The miR-7 microRNA can be isolated, synthetic or recombinant.

Precursors or derivatives of miR-7 microRNAs include pre-miRNA precursors that can be processed by the RNAi machinery of a cell into a mature miRNA which can mimic the activity of mature miR-7.

In some embodiments, the miR-7 microRNA is provided in the form of a synthetic double-stranded RNA molecule, comprising a mature miR-7 sequence on a first strand and a sequence complementary to the mature miR-7 sequence on a second strand (also known as the passenger stand).

Double stranded RNA generally includes first and second strands of RNA having sufficient sequence complementarity between the strands to enable the first and second strands to bind to each other by Watson-Crick base pairing to form a ‘stem’. In some embodiments, a stem of dsRNA consists of 100% complementarity between the first and second strands. In some embodiments, there may be one or more mismatches between the first and second strands forming the stem so that the complementarity between strands of the stem may be less than 100%. Generally the level of complementarity between first and second strands of the stem is greater than 80%, preferably 85%, preferably 90%, preferably 95, 96, 97, 98 or 99%. Generally, the mismatches are across a contiguous sequence of no more than about 3 nucleotides, preferably about 2 nucleotides. Preferably a mismatch is limited to between single nucleotides of the first and second strands of the stem at spaced apart locations.

Given above, it will be understood that a stem of dsRNA of defined length may in fact include one or more regions or positions of mismatch. For example, where first and second strands have perfect complementarity across a region of 30 nucleotides but for one or two mismatches within the 30 nucleotide region, the first and second strands would be said to constitute a stem of dsRNA of 30 nucleotides.

A dsRNA may include one or more stems. Where there is more than one stem, these may be arranged in series or clusters to form tandem or overlapping inverted repeats, which form dsRNA structures resembling, for example, a two-stem structure resembling a “hammerhead”, “barbell”, or “dog bone”, or a structure containing 3 or more stems resembling a “cloverleaf”, or a structure with a pseudoknot-like shape. Any of these constructs can further include spacer segments found within a double-stranded stem (for example, as a spacer between multiple anti-sense or sense nucleotide sequence segments or as a spacer between a base-pairing anti-sense nucleotide sequence segment and a sense nucleotide sequence segment) or outside of a double-stranded stem (for example, as a loop region of sense or of anti-sense or of unrelated RNA sequence, separating a pair of inverted repeats). In cases where base-pairing anti-sense and sense nucleotide sequence segment are of unequal length, the longer segment can act as a spacer.

In an alternative embodiment, the mature miR-7 sequence is provided as a single-stranded RNA molecule. For example, the double stranded RNA molecule may arise from a single strand of RNA having repeat sequences that enable the single strand of RNA to form a stem structure. Alternatively, the double strand RNA molecule may arise from two RNA molecules that align and forms base pair bonds with each other to form a double stranded RNA molecule (or duplex).

The present invention also contemplates the use of RNA molecules which can act as a precursor of the mature miR-7 sequence. In other words, the present invention relates to the use of a miRNA mimic such that upon provision to the cell, the precursor is recognised by the different components of the cell's RNAi machinery and processed such that the RNA molecule which ultimately forms part of the miRISC complex, can mimic the behaviour of miR-7-5p. In certain embodiments, the invention includes the provision of double-stranded RNA molecules which have the same or similar sequence and structural characteristics as pre-miR-7-1, pre-miR-7-2 and pre-miR-7-3.

Isolated, means a miR-7 gene product that is synthesised, or altered, or removed from the natural state through human intervention. For example, a synthetic miR-7 gene product or a miR-7 gene product partially or completely separated from the coexisting materials of its natural state in a cell, for example, is considered to be “isolated.” An isolated miR-7 gene product can exist in a substantially purified form, or it can exist in a cell into which the miR-7 coding sequence or product has been introduced. Thus, a miR-7 gene product (such as a pre-miR-7 or mature miR-7-5p molecule) that is deliberately to, or expressed on a cell is considered an “isolated” product. A miR-7 gene product produced inside a cell from a miR precursor molecule is also considered to be an “isolated” molecule.

The isolated, recombinant or synthetic molecules described herein can be used in a method of treating ACC and can also be used in the manufacture of a medicament for the treatment of ACC.

Isolated miR-7 microRNA molecules can be obtained using any number of standard techniques familiar to the skilled person. For example, the miR-7 molecule can be chemically synthesised or recombinantly produced using methods well within the purview of the skilled person. In one embodiment, the miR-7 molecule is chemically synthesised using appropriately protected ribonucleoside phosphamidites and a conventional DNA/RNA synthesizer. Commercial suppliers of synthetic RNA molecules and synthesis reagents can also be used and include: Proligo, Dharmacon Research, Pierce Cehmical, Glen Research, ChemGenes, Ambion, Life Technologies and others.

### Nucleic Acid Constructs

The miR-7 molecule can also be a recombinant molecule expressed from recombinant circular or linear DNA constructs using any suitable promoter. Accordingly, the present invention relates in part to providing nucleic acid constructs which encode a miR-7 precursor or derivative, such that the miR-7 molecule is transcribed in situ in the adrenocortical carcinoma cell.

In a further embodiment, the present invention relates to a nucleic acid construct for encoding a miR-7 microRNA, wherein the nucleic acid construct includes:
a coding region for encoding a double stranded RNA molecule;
a promoter operable in a cell for the production of a double stranded RNA molecule;
wherein the double stranded RNA molecule has a sequence that enables the production of a miR-7 microRNA.

Suitable promoters for expressing RNA from a plasmid include, for example, the U6, H1 RNA Pol II promoter sequences or the cytomegalovirus promoters. Selection of other suitable promoters is within the skill set of the person of skill in the art. The recombinant plasmid constructs of the invention may also comprise inducible or regulatable promoters for expression of miR-7 gene products in cancer cells.

“Enables the production of” means that the double stranded RNA molecule can be any RNA molecule which can be processed by the RNAi machinery of a cell to form a mature miR-7 duplex comprising an active strand and a passenger strand. The active strand will have a sequence which is at least 80% identical to the sequence of SEQ ID NO: 2. For example, the double stranded RNA molecule can be formed from a single RNA transcript encoded by the nucleic acid construct. As described above, single-stranded RNA molecules, if they have regions of inverted repeat sequence, may form stem and loop structures such that they behave as a double-stranded RNA molecule, which can then be recognised by the RNAi machinery of a cell, and processed into a short double-stranded RNA duplex recognised and bound by the RISC complex.

miR-7 gene products that are expressed from recombinant nucleic acid constructs can also be delivered to and expressed directly in the cancer cells. Alternatively, the miR-7 products that are expressed from the recombinant constructs can be isolated from a cultured cell expression system using standard techniques.

The skilled person will be familiar with the selection of various plasmids suitable for expression of a recombinant miR-7 microRNA including methods for inserting nucleic acid sequences into the constructs to express a relevant product and methods for delivering the recombinant plasmid construct to the cells of interest, such as adrenocortical carcinoma cells.

The miR-7 microRNAs for use in accordance with the methods of the present invention may also be expressed from recombinant viral vectors. Any viral vector capable of accepting the coding sequences for the miR-7 gene products can be used, for example, vectors derived from adenovirus (AV), adeno-associated virus (AAV); and retroviruses such as lentivirus.

### Chemical Modifications

The present invention also contemplates the use of structurally and chemically modified double-stranded RNA molecules in order to improve the efficiency of targeting of the miR-7 molecule to its target. For example, in certain exemplary embodiments, non-toxic chemical modifications to the miRNA sequence have been introduced to improve stability, reduce off-target effects and increase activity.

In one embodiment, the miRNA molecule includes an RNA duplex comprising the mature miR-7 sequence and a passenger strand. In one aspect, the passenger strand is structurally and chemically modified to enable the retention of activity of the duplex while inactivating the passenger strand, thereby reducing off-target effects. In a further aspect, chemical modification inhibits nuclease activity, thereby increasing stability.

In further embodiments, the miRNA molecules of the invention include nucleotides that are modified to enhance their activities. Such nucleotides include those that are at the 5′ or 3′ terminus of the RNA molecule as well as those that are not located at the termini of the molecule. Modified nucleotides used in the complementary strands of miRNA either block the 50H or phosphate of the RNA or introduce internal sugar modifications that prevent uptake and activity of the inactive strand of the miRNA. Modifications for the miRNA include internal sugar modifications that enhance hybridization as well as stabilize the molecules in cells and terminal modifications that further stabilize the nucleic acids in cells. Further contemplated are modifications that can be detected by microscopy or other methods to identify cells that contain the microRNAs.

In other aspects, modifications may be made to the sequence of a miRNA or a pre-miRNA without disrupting microRNA activity. As used herein, the term “functional variant” of a miRNA sequence refers to an oligonucleotide sequence that varies from the naturally-occurring miRNA sequence, but retains one or more functional characteristics of the miRNA (e.g., enhancement of cancer cell susceptibility to chemotherapeutic agents, cancer cell proliferation inhibition, induction of cancer cell apoptosis, specific miRNA target inhibition). In some embodiments, a functional variant of a miRNA sequence retains all of the functional characteristics of the miRNA. In certain embodiments, a functional variant of a miRNA has a nucleobase sequence that is a least about 60%, 65%, 70%, 75%, 80%, 85%, 90%, 91%, 92%, 93%, 94%, 95%, 96%, 97%, 98% or 99% identical to the miRNA or precursor thereof over a region of about 5, 6, 7, 8, 9, 10, 1 1, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases, or that the functional variant hybridizes to the complement of the miRNA or precursor thereof under stringent hybridization conditions. Accordingly, in certain embodiments the nucleobase sequence of a functional variant may be capable of hybridizing to one or more target sequences of the miRNA.

In some embodiments, the complementary strand is modified so that a chemical group other than a phosphate or hydroxyl is at its 5′ terminus. The presence of the 5′ modification is thought to eliminate uptake of the complementary strand and subsequently favours uptake of the active strand by the RISC complex. The 5′ modification can be any of a variety of molecules known in the art, including NH₂, NHCOCH₃, and biotin.

In another embodiment, the uptake of the complementary (passenger) strand by the RISC complex is reduced by incorporating nucleotides with sugar modifications in the first 2-6 nucleotides of the complementary strand. It should be noted that such sugar modifications can be combined with the 5′ terminal modifications described above to further enhance miRNA activity. Sugar modifications contemplated in miRNA mimics include, but are not limited to, a sugar substitute group selected from: F; 0-, S-, or N-alkyl; 0-, S-, or N-alkenyl; 0-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. In some embodiments, these groups may be chosen from: O(CH₂)ₓOCH₃, O((CH₂)ₓO)_{y}CH₃, O(CH₂)ₓNH₂, O(CH₂)—CH₃, O(CH₂)—ONH₂, and O(CH₂)—ON((CH₂)xCH₃)₂ where x and y are from 1 to 10.

Altered base moieties or altered sugar moieties also include other modifications consistent with the purpose of a miRNA. Such oligomeric compounds are best described as being structurally distinguishable from, yet functionally interchangeable with, naturally occurring or synthetic unmodified oligonucleotides. As such, all such oligomeric compounds are contemplated to be encompassed by this invention so long as they function effectively as a miR-7 molecule, i.e., a miRNA molecule having the sequence of SEQ ID NO: 1.

In some embodiments, the complementary strand is designed so that nucleotides in the 3′ end of the complementary strand are not complementary to the mature miR-7 sequence. This results in double-stranded hybrid RNAs that are stable at the 3′ end of the active strand but relatively unstable at the 5′ end of the active strand. This difference in stability enhances the uptake of the active strand by the microRNA pathway, while reducing uptake of the complementary strand, thereby enhancing microRNA activity.

Other modifications, contemplated for use in the practice of the invention, include the provision of a miR-7 miRNA duplex comprising (i) a sense strand that ranges in size from about 16 to about 31 nucleotides in which about 40% to about 90% of the nucleotides of the sense strand are chemically modified; (ii) an antisense strand that ranges in size from about 16 to about 31 nucleotides in which about 40% to about 90% of the nucleotides of the antisense strand are chemically modified nucleotides; and (iii) at least one of a mismatch between nucleotide 1 on the antisense strand and the opposite nucleotide on the sense strand; and a mismatch between nucleotide 7 on the antisense strand and the opposite nucleotide on the sense strand. Also advantageous in this context, as disclosed, is the attachment to the sense strand of the miRNA duplex, via a linker molecule that is from about 3 to about 9 atoms in length, of a conjugate moiety selected from the group consisting of cholesterol, cholestanol, stigmasterol, cholanic acid, and ergosterol. The linker molecule can be 5 to 8 atoms in length, for example, and the linker molecule can attach the conjugate moiety to the 3′ end of the sense strand.

Chemical modifications used in accordance with the present invention include phosphorothioate containing oligonucleotides, 2′-O-methyl-(2′-O-Me) or 2′-O-methoxyethyl-oligonucleotides (2′-O-MOE-), locked nucleic acid (LNA) oligonucletoides, peptide nucleic acids (PNA), fluorine derivatives (FANA and 2′F) and other chemical modifications known to the skilled person.

A nucleic acid may be made by any technique known to one of ordinary skill in the art, such as for example, chemical synthesis, enzymatic production or biological production. In some embodiments, microRNA compositions of the invention are chemically synthesized.

### Compositions and Methods of Administration

In accordance with the methods of the present invention, the miR-7 microRNA can be administered directly to an individual requiring miR-7 microRNA therapy or in the cells in which miR-7 microRNA therapy is required.

In a further embodiment, the invention includes indirect administration of miR-7 microRNA whereby a nucleic acid construct which encodes a miR-7 microRNA is provided in ACC cells, such that miR-7 microRNA can be transcribed endogenously within the ACC cell.

The present invention thus relates to agents and pharmaceutical compositions for delivery of miR-7 microRNAs in the individual or in the specific cells in which miR-7 therapy is required.

In general, suitable agents and compositions may be prepared according to methods which are known to those of ordinary skill in the art and may include a pharmaceutically acceptable diluent, adjuvant and/or excipient.

In one embodiment, the present invention relates to an agent in the form of a nanoparticle having a miR-7 molecule attached thereto. The miR-7 molecule may be wholly contained within, attached or adhered to the surface of the nanoparticle. The agent may be adapted for systemic administration or for direct administration into the cancer growth.

The nanoparticle may be any vesicle which can be taken up by a cell (for example by phagocytosis or endocytosis) such that the contents of the vesicle are provided into the cytoplasm of the cell.

The anti-cancer agent may be adapted for targeted delivery of miR-7 to specific cells and tissue types. This can be accomplished, for example, with the use of antibodies or antigen-binding fragments on the surface of the nanoparticles being used to encapsulate the miR-7 molecules. The antibodies preferably, would have affinity for receptors found on the surface of the cells to which the miR-7 is to be provided. In the case of ACC cells, the antibody may have specificity to Epidermal Growth Factor Receptor (EGFR), which is expressed on the surface of adrenocortical carcinoma cells. Alternatively, the antibody could be once which binds to Insulin-like Growth Factor 2 (IGFR2), also highly expressed on the cell surface of ACCs. Where the cancer is located elsewhere in the body, an alternative antibody may be selected. For example, for targeting to breast tissue, an anti-HER2 antibody or antigen-binding fragment may be selected.

In an exemplary embodiment, the miR-7 molecules described herein are administered to individuals requiring treatment, using intact, bacterially derived minicells. In a particularly preferred embodiment, the mir-7 molecule is delivered via the “EnGeneIC Delivery Vehicle” system developed by EnGeneIC Molecular Delivery Pty Ltd (Sydney), which is based on the use of intact, bacterially derived minicells. The EDV™ system is described, for example, in published international applications WO 2006/021894 and WO 2009/027830, the respective contents of which are incorporated here by reference.

In the context of using bacterially-derived minicells, it is possible to use a bispecific antibody for targeting the minicells to target tissues. One moiety of such an antibody has specificity for the target tissue, while the other has specificity for the minicell. The antibody brings minicells to the target cell surface, and then the minicells are brought into the cell by endocytosis. After uptake into the tumor cell there is a release of the minicell contents, i.e., the miR-7(s).

Other methods of preparing nanoparticles for delivery of miR-7 microRNA will be familiar to the skilled person. For example, the miR-7 microRNA may be administered using liposomes, synthetic polymeric materials, naturally occurring polymers and inorganic materials to form nanoparticles. Examples of lipid-based materials for delivery of the RNA molecules include: polycationic liposome-hylauronic acid (LPH) nanoparticles, DOTMA:cholesterol:TPGS lipoplexes, dicetyl phosphate-tetraethylemepentamine-based polycation liposomes (TEPA-PCL, and neutral lipid emulsions (NLEs).

Examples of synthetic polymeric materials which can be used to deliver the miR-7 microRNA include polyethylenimine (PEI)-based delivery systems, including polyurethane-short branch polyethylenimine (PU-PEI) carriers, and dendrimers including poly(amidoamine) (PAMAM) dendrimer, poly(lactide-co-glycolide) (PLGA) particles. Naturally occurring polymers which can be used for form nanoparticles for encapsulating the miR-7 molecules include chitosan, protamine, atelocollagen and peptides.

Inorganic materials may also be used in accordance with the invention to produce nanoparticles for providing the miR-7 RNA molecules to the cells. For example, gold nanoparticles, silica-based, and magnetic nanoparticles for delivery of the miR-7 RNA molecules may be produced by methods known to the person skilled in the art.

The present invention also contemplates the use of the above-described means for providing a nucleic acid construct encoding miR-7 micro in the ACC cells. For example, the nanoparticle described above may include a nucleic acid construct such as a viral construct, which, once taken up by the ACC cell, drives transcription of miR-7 microRNA from the vector construct using the transcription machinery of the cell.

The agents and compositions described herein may be administered via any convenient or suitable route. For example, the route of administration may be parenteral (e.g., intraarterial, intravenous, intramuscular, subcutaneous), oral, nasal, mucosal, intracavitary or topical. The compositions may be formulated in a variety of forms including solutions, suspensions, emulsions and solid forms, and are typically formulated so as to be suitable for the chosen route of administration, for example as capsules, tablets, elixirs for oral ingestion, in an aerosol for administration by inhalation, ointment, cream, gel, jelly or lotion suitable for topical administration or an injectable formulation suitable for parenteral administration. The preferred route of administration will depend on a number of factors and the preferred outcome. In one embodiment, the nucleic acids in accordance with the present invention may be administered via intratumoural injection.

### Dosing

As used herein, an “effective amount” of a nucleic acid or RNA molecule is the amount that is required to treat one or more symptoms of cancer, in particular, adrenocortical cancer, reverse the progression of one or more symptoms of the cancer, halt the progression of one or more symptoms of the cancer, or prevent the occurrence of one or more symptoms of the cancer in a subject to whom the RNA molecule is administered. In a particularly preferred embodiment, an effective amount is one which is sufficient to inhibit the proliferation and/or cell cycle of a cancer cell in a subject.

One skilled in the art can readily determine an effect amount of a miR-7 microRNA molecule or composition to be administered, by taking into account factors such as size and weight of the subject, the extent of disease penetration, the age, health and sex of the subject, the route of administration and whether the administration is regional or systemic.

In some embodiments, the amount of miR-7 microRNA provided is one that is sufficient to ensure that the amount of miR-7 achieved in the cancer cells, is greater than the amount seen in the neoplastic cell. For example, in the context of adrenocortical carcinoma, and without wishing to be bound by theory, inventors believe that miR-7 acts in a tumour-suppressive manner, whereby reduced levels of miR-7 seen in cancer cells relieves the suppressive activity and contributes to the progression and/or development of the neoplasm.

In yet further embodiments, the effective amount is one whereby a steady-state level of miR-7 microRNA in the ACC is achieved. Steady-state means an amount of miR-7 molecules in the cell, sufficient to “restore” normal miR-7 tumour-suppression activity to the levels observed in non-neoplastic adrenal cortex cells.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Example 1—Determination of miR-7 Levels in ACC and miR-7 Therapy in ACC

### Materials and Methods

### Clinical Samples

Ethics approval was obtained from the Northern Sydney Area Health Service Human Research Ethics Committee and informed consent was obtained from all patients whose samples were used in this study. ACC and normal adrenal cortex (NAC) tissue samples were obtained during surgery, snap frozen in liquid nitrogen and stored at −80° C. in the Neuroendocrine Tumor Bank of the Kolling Institute of Medical Research. The diagnosis of all samples was confirmed by centralized pathological review by an experienced endocrine pathologist.

### Cell Culture and Transfections

The human ACC cell line NCI-H295R (H295R) and SW13 were 356 purchased from the American Type Culture Collection (ATCC, VA, USA). H295R cells (ATCC CRL-2128) were cultured in DMEM/F12 (Life Technologies, CA, USA) supplemented with 5% fetal bovine serum (FBS) and 91% ITS+ Premix supplement (BD Biosciences, MA, USA). SW-13 cells (ATCC CCL-105) were cultured in Leibovitz's medium (Life Technologies) supplemented with 10% FBS. Cells were cultured at 37° C. in a humidified atmosphere under 5% CO2. The cell lines were negative in periodic monitoring for mycoplasma and independently genotyped to rule out cross-contamination by Cell Bank Australia (Westmead, Australia). Cells were transfected with a final concentration of 40 nM of synthetic miRNA mimics (mirVana miRNA mimics, Cat No. 4464066 Life Technologies) corresponding to hsa-miR-7-5P (Product ID: MC10047) or a negative control miRNA (miR-NC: Product ID: AM171100) using Lipofectamine RNAiMax (Life Technologies) according to the manufacturer's protocol. For transfections, cells were plated in six-well plates and transfected when they were 50-70% confluent and three days later a second transfection was performed. Cells were collected for down-stream analysis three days after the second transfection.

### Cell Proliferation and Cell Cycle Analysis

Cell proliferation was performed using CellTiter 96 Aqueous One Solution Cell Proliferation Assay according to the manufacturer's instruction (MTS Assay, Promega, Wis., USA). For this assay, 5,000 cells were measured and cultured per well in a 96-well plate. Transfection of microRNA mimics were performed at the same time (measured as day 0) and proliferation data, using absorbance was measured on day 1 to day 5 following transfection. Absorbance at 490 nm was measured using a 96-378 well plate reader (Sunrise microplate reader, Tecan, Switzerland). For cell cycle analysis, 2.5×105 H295R or SW-13 cells were cultured in a 6-well plate. Cells were collected, washed with Phosphate Buffered Saline (PBS) and stained with Propidium Iodide (PI, Sigma Aldrich) at a final concentration of 17.4 μg/ml. Cells were analysed using fluorescence-activated cell sorting (FACS) analysis (FACS Calibre, BD Biosciences) and flow cytometry histograms were modelled using Modfit LT software (Verity Software House, ME, USA).

### RNA Extraction and RT-qPCR

Total RNA was extracted from frozen tumor samples and ACC cells using the miRNeasy Mini Kit (Qiagen, Hilden, Germany). RNA concentration and quality was assessed using a NanoDrop ND 1000 Spectrophotometer (ThermoFisher Scientific, MA, USA) and an Agilent 2100 Bioanalyzer (Agilent Technologies, CO, USA). The expression levels of individual miRNAs were measured with quantitative reverse transcription-polymerase chain reaction (RT-qPCR) using Taqman miRNA assays (Life Technologies) according to the manufacturer's instructions. Briefly, 10 ng of total RNA was first reverse transcribed to complementary DNA (cDNA) using TaqMan miRNA primers and the PCR products were then amplified from cDNA and quantified with the ABI 7900HT Real-time PCR System (Applied Biosystems) under standard cycling conditions. Relative expression (RQ) was obtained using the ΔΔCₜ method and the differences between groups were assessed using DataAssist Version 3.01 (Applied Biosystems). RNU48 was used as a reference gene for human and xenograft samples and mouse U6 snRNA for mouse organs. Samples across all PCR plates were calibrated against commercially available human adrenal cortex total RNA (Clontech, CA, USA). For the measuring of mRNA expression levels, 1 μg of total RNA was reversed transcribed using the high capacity RNA-to-cDNA reverse transcription kit (Life Technologies) and the PCR was amplified using standard TaqMan gene expression assays (Life Technologies). GAPDH was used as a reference gene for human and xenograft samples and mouse B2m for mouse organs.

### Protein Extraction and Immunoblotting

Cells were lysed using Radio-Immunoprecipitation Assay (RIPA) Buffer and protein concentration was quantified using Pierce BCA Protein Assay Kit (Pierce Biotechnology, IL, USA). 30 μg of protein lysate was denatured at 70° C. for 10 min before electrophoresis on precast 4-12% bis-Tris gels (Life Technologies). Separated proteins were transferred to Immobilin P membranes (Merck Millipore, MA, USA). The membranes were blocked in Tris-buffered saline with 0.1% Tween-20 (TBST) containing 5% BSA and probed with the antibody of interest. The Western Bright Quantum detection kit (Advansta, CA, USA) was used to visualize the detected proteins by a LAS4000 digital imaging system (Fujifilm, Tokyo, Japan). Protein loading was normalized to GAPDH and expression quantified using MultiGauge software (V 3.0, Fujifilm) and mean expression was calculated from three experiments.

### Luciferase Reporter Assays

Human genomic DNA was used to amplify the 3′ UTR of miRNA target genes by PCR and the amplified PCR fragment was cloned into the pMIR-REPORT Luciferase Vector (Part Number AM5795, Life Technologies) between SpeI and SacI restriction sites. For the EGFR-3′ UTR reporter, primers 5′-GACTACTAGTCTTCAATGGGCTCT TCCAACAAGG-3′ and 5′-GACTGAGCTCGGTCCAAATGCTGATGAATCC-3′ were used to amplify a fragment of 532 bp containing two predicted miR-7 seed binding sequences. For the RAF1-3′ UTR reporter, primers 5′-GACTACTAGTGAAGTAAGGTAGCAGGCAGTCC-3′ and 5′-GACTGAGCTCTGAGGGACCATCAGATAACTG-3′ were used to amplify a fragment of 555 bp also containing two seed binding miR-7 target sequences. ACC cells were co-transfected with the pMIR Luciferase Reporter Vector plus Renilla Luciferase Control Vector (Promega) along with the miRNA using Lipofectamine 2000 (Life Technologies) and the luciferase activity was quantified using the Dual-Luciferase Reporter Assay System (Promega) using a luminometer (Veritas Microplate Luminometer, Turner Biosystems, CA, USA). Relative luciferase activity was quantified by calculating the firefly to Renilla luciferase signal ratio.

### EDV™ Nanocell Preparation

miRNAs were packaged into the EDVs for systemic delivery using a method of diffusion with overnight incubation previously reported for siRNA loading [10]. Following miRNA loading, EDVs were incubated with 5 μg of a bispecific monoclonal antibody (BsAb) against human epidermal growth factor receptor (EGFR) for an hour at 24° C. as previously reported [10]. The ensuing product was named ^{EGFR}EDV™ _{miRNA} (e.g. ^{EGFR}EDV™ _{miR-7}).

### Mouse Xenografts

Protocols for xenograft experiments in female athymic (nu/nu) mice 445 (4-6 weeks old) were approved by the EnGeneIC Animal Ethics Committee. H295R cells (1×10⁷ cells in 100 μl serum free DMEM/F12K medium) with 100 μl BD Matrigel basement membrane matrix-growth factor reduced, phenol red free (BD Biosciences), which contains less than 0.5 ng/ml of epidermal growth factor, were inoculated subcutaneously into the left flank of each nude mouse. Patient-derived xenografts were established with inoculation of the same number of primary ACC cells isolated from ACC surgical tumor samples using the same protocol. Mice were randomized to six mice per group to receive a control: scrambled miRNA sequences (^{EGFR}-EDV™ _{miR-NC}) or treatment: miR-7 (^{EGFR}EDV™ _{miR-7}) when tumours reached ˜100 mm³. Mice were treated four times per week by tail vein injection. Experiments ended when a significant difference between the treatment and control groups was detected according to the ethics protocol. Initial sample size was estimated by assuming a difference of 30% in tumour size between the control and treatment groups, with statistical significance of less than 0.05, a minimum of six animals were needed for greater than 90% power.

### Histopathology & Immunohistochemistry

Following euthanasia, the tumours and organs were excised and flash frozen in liquid nitrogen and stored at −80° C. Histopathology on formalin fixed samples following H&E staining and immunohistochemistry was performed by a pathologist blinded to the treatment group. For EGFR staining, immunohistochemistry was scored semi-quantitatively from 0 (negative), to 1+(focally or weakly positive) to 2+(moderate staining) to 3+(diffuse strong staining).

### Statistics

Statistics were calculated using Prism Software Version 6.0 (GraphPad, CA, USA) using Students t-test for parametric data and Mann-Whitney test for nonparametric data. Differences in gene expression were assessed by t-test using DataAssist Version 3.01 (Applied Biosystems). Statistical significance was set as P<0.05.

### Results

miR-7 is Under-Expressed in ACC Clinical Samples

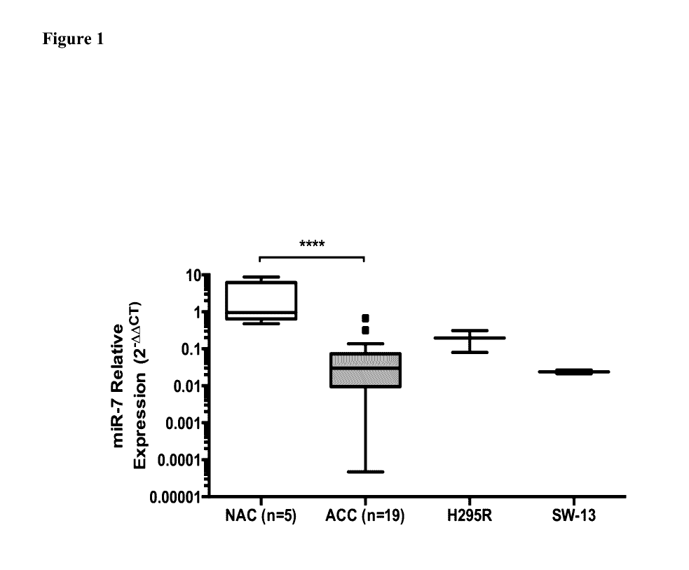

RT-qPCR was performed on a group of 19 ACC and 5 NAC clinical samples. This analysis showed that miR-7 was significantly reduced in ACC compared to NAC with a fold change of 0.04 or a 25-fold reduced expression of miR-7 in ACC (FIG. 1). ACC has two established cells lines available (H295R and SW-13) both of which also had reduced miR-7 expression compared to the NAC samples (FIG. 1). H295R cells are hormone producing (functional) and the best characterized in study of the ACC, while SW-13 are non-hormone producing and while derived from an adrenal surgical sample it is not clear whether they arose from a primary ACC or metastasis [11]. For this study, SW-13 cells were used as secondary cell line to investigate miR-7 action in a non-functional ACC model.

miR-7 Inhibits Cell Proliferation and Induces Cell Cycle Arrest

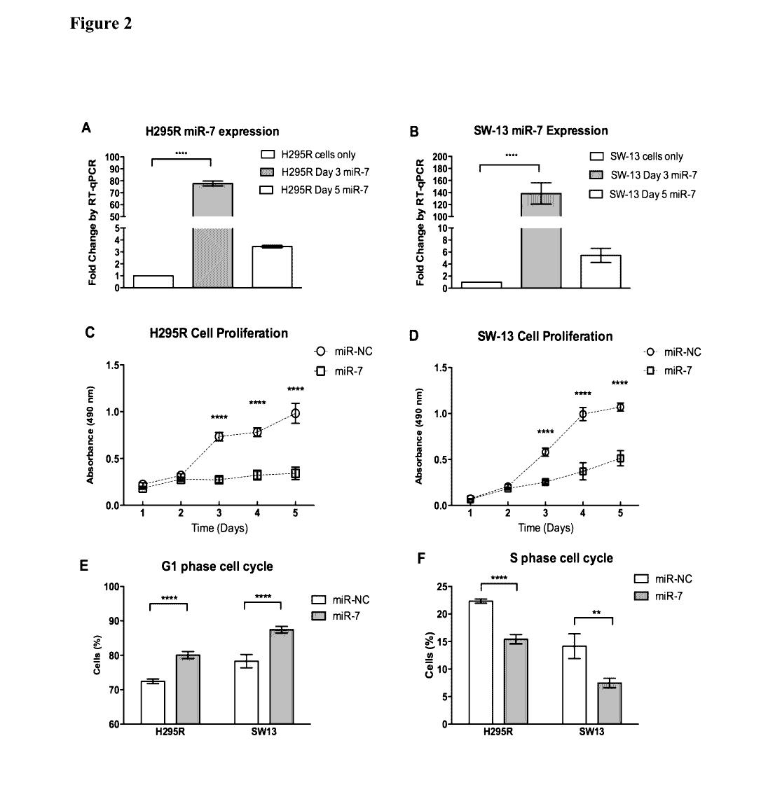

To explore the role of endogenous miR-7 in the pathogenesis of ACC, miR-7 was overexpressed in the H295R and SW-13 cells and the cell phenotypes induced were studied using scrambled miRNA sequences (miR-NC) as a negative control. Following transfection, increased miR-7 expression was confirmed by RT-qPCR (FIG. 2A, 2B). Over-expression of miR-7 resulted in significant inhibition of cell proliferation in both H295R and SW-13 cells (FIG. 2C, 2D). The cause of the reduction in cell proliferation following miR-7 overexpression was explored using analysis of cell division and cell death. Cell division analysis using flow cytometry showed cells transfected with miR-7 had a significantly reduced percentage in S phase and an increase in G₁ phase when compared to miR-NC transfected cells (FIG. 2E, 2F). Cell death analysis using apoptosis assays revealed no significant changes in the cell population transfected with miR-7 vs. miR-NC. From these results, it is proposed that miR-7 in ACC acts in vitro to reduce cell proliferation by inducing G1 cell cycle arrest.

RAF1 and MTOR are Reduced Following miR-7 Replacement in ACC Cell Lines

To investigate the mechanisms through which miR-7 may act as a tumor suppressor, the predicted targets of miR-7 were examined using four prediction algorithms; DIANA-microT-CDS v5.0 (B.S.R.C. Alexander Fleming, Athens, Greece), DIANA-miRPath v2.0 (B.S.R.C. Alexander Fleming), TargetScan (Whitehead Institute for Biomedical Research, MA, USA) and miRanda (Welcome Trust Sanger Institute, UK). The analysis of DIANA-miRPath v2.0 predicted that mammalian target of rapamycin (mTOR) signalling pathway as the top pathway targeted by miR-7 with 10 genes, including mechanistic target of rapamycin (MTOR) and eukaryotic translation initiation factor 4E (EIF4E). A list of predicted targets from each database, which encode pivotal components of key cancer-related signalling pathways were compared and intersected to narrow the list of potential genes to higher confidence targets. Targets selected included Raf-1 proto-oncogene serine/threonine kinase (RAF1) and Epidermal Growth Factor Receptor (EGFR) as key genes in the mitogen-activated protein kinase pathway (MAPK) signalling pathway and MTOR and EIF4E involved in the mTOR pathways.

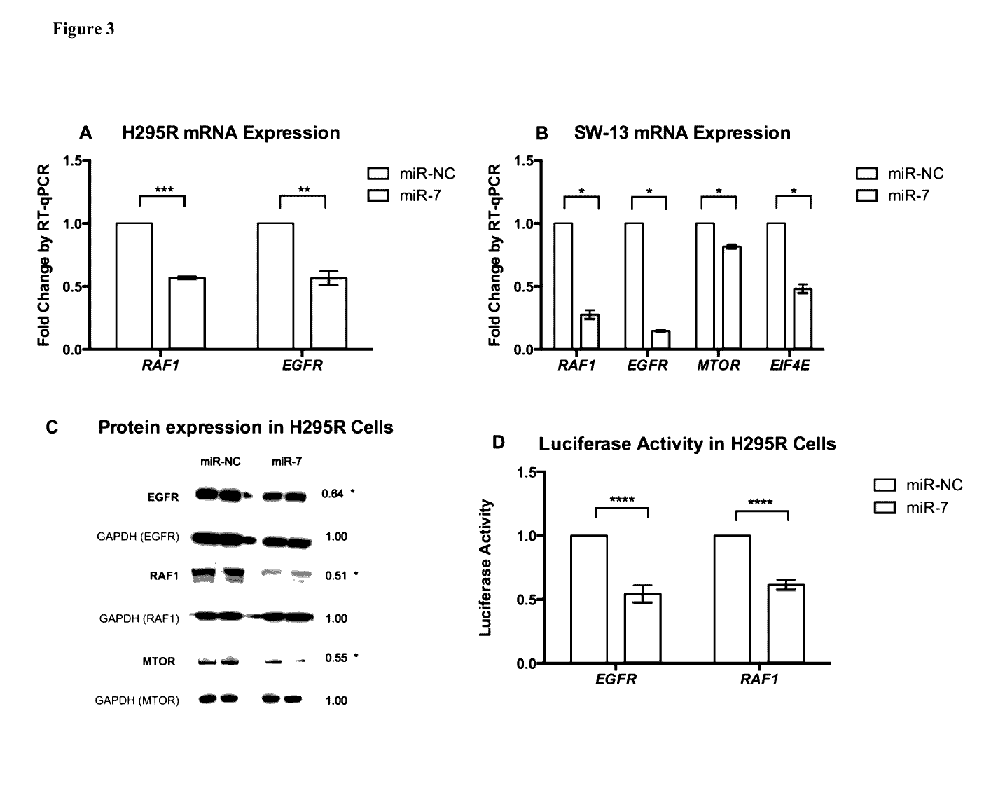

To determine whether miR-7 repressed any of these putative targets, cells were transfected and mRNA levels assessed by RT-qPCR. Over-expression of miR-7 significantly reduced expression of RAF1 and EGFR in both H295R and SW-13 ACC cell lines (FIG. 3A, 3B), while MTOR and EIF4E was significantly reduced in SW13 cells only (FIG. 3B). Reduced expression of RAF1, EGFR and MTOR protein by miR-7 was detected by Western blotting in H295R cells (FIG. 3C).

Both RAF1 and EGFR contain two predicted seed binding sites in their 3′ UTRs that are highly conserved in mammals. To verify that these transcripts are directly regulated by miR-7, the 3′ UTR sequences of EGFR and RAF1, encompassing the predicted seed binding sites that would disrupt miRNA interaction, were inserted into the multiple cloning site of the pMIR145 REPORT miRNA Expression Reporter Vector (Life Technologies). Co-transfection of the reporter vector with miR-7 mimic or miR-NC was performed in H295R cells. Co-transfection with miR-7 suppressed luciferase activity of both the RAF1 and EGFR reporters (FIG. 3D) when compared with that co-transfected with miR-NC, confirming the seed binding sequences of miR-7 on the 3′ UTR region of RAF1 and EGFR genes. Taken together, these results indicate miR-7 acts as a tumor suppressor in ACC by affecting multiple molecular targets, involved in the mTOR and MAPK signalling pathways.

miR-7 Therapy Using EDV Nanoparticles Reduces ACC Xenograft Growth

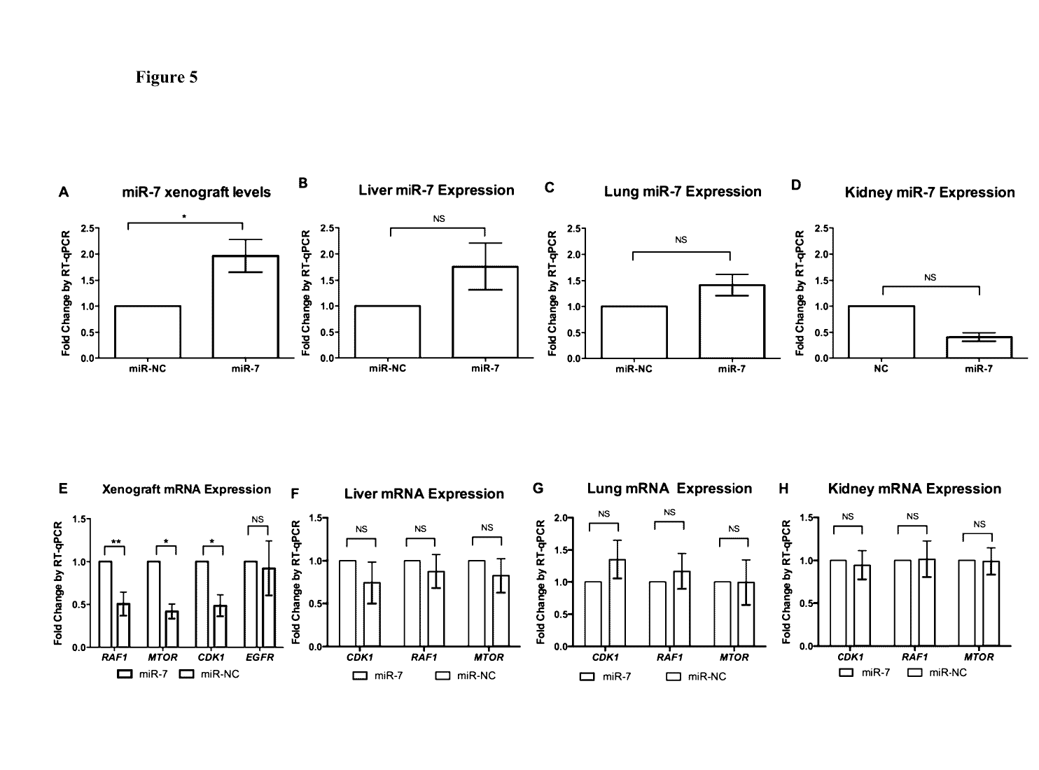

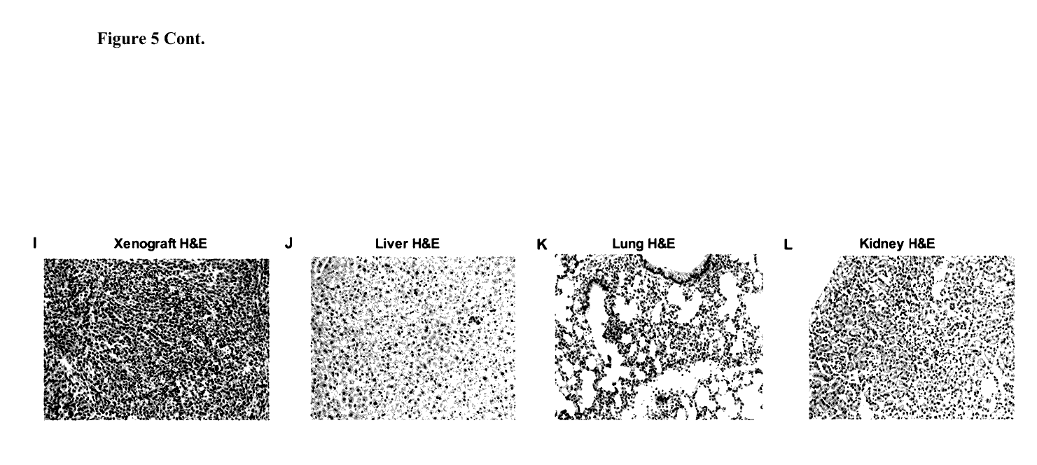

Having demonstrated that miR-7 arrests proliferation of ACC cells in vitro, a series of in vivo experiments were initiated to assess whether targeted delivery by intravenous injection of miR-7 using EDV nanoparticles could be used as a therapeutic for ACC. H295R and patient-derived xenograft models were established and miR-7 mimic was delivered using the targeted nanoparticle delivery system—^{EGFR}EDV™ nanocells [12]. EGFR targeted nanoparticles were used as EGFR is expressed in ACC (FIG. 5A).

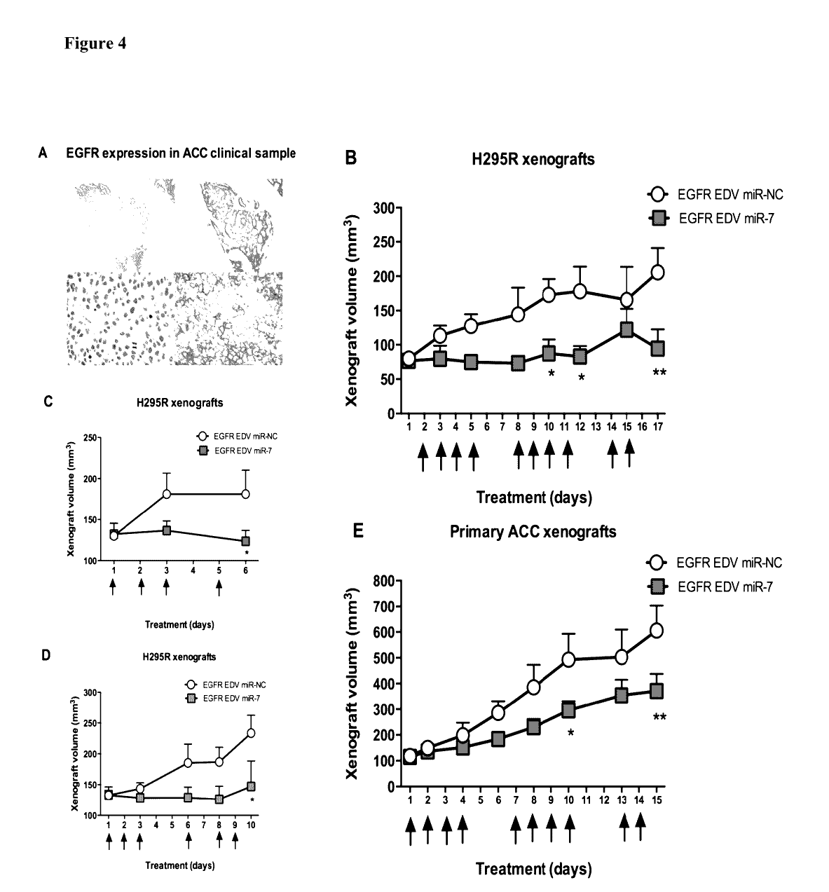

For the initial H295R xenograft experiment, miR-7 and miR-NC was intravenously administered by tail vein injection at a dose of 2×10⁹ EDVs containing 0.32 nmoles of either miR-7 or miR-NC in ten doses over a two-week period. After 17 days, ACC tumor volume had increased by over two fold in the miR-NC treated group and remained unchanged in the miR-7 treated group (FIG. 4B). To confirm these findings and to investigate the molecular target knock down of miR-7 regulation of molecular targets, H295R xenografts were established on two further separate occasions and tumors were collected following four and six doses of treatment. miR-7 therapy demonstrated tumor reduction as early as two doses of treatment and similar tumor inhibition effect was seen with each experiment (FIG. 4C, 4D).

To further test the use of miR-7 replacement as a therapeutic, this regimen was tested in a patient derived xenograft. ACC primary cells were isolated from an ACC surgical sample and inoculated subcutaneously. Systemic delivery of ten doses of miR-7 in this xenograft model showed significant tumor reduction in the miR-7 group vs miR-NC group at the end of the treatment period (FIG. 4E). Taken together, this demonstrates that systemic targeted miR-7 replacement using a nanoparticle delivery system inhibits ACC growth in both cell line and patient-derived xenografts.

Systemic miR-7 Therapy In Vivo Leads to Inhibition of RAF1, MTOR and CDK1

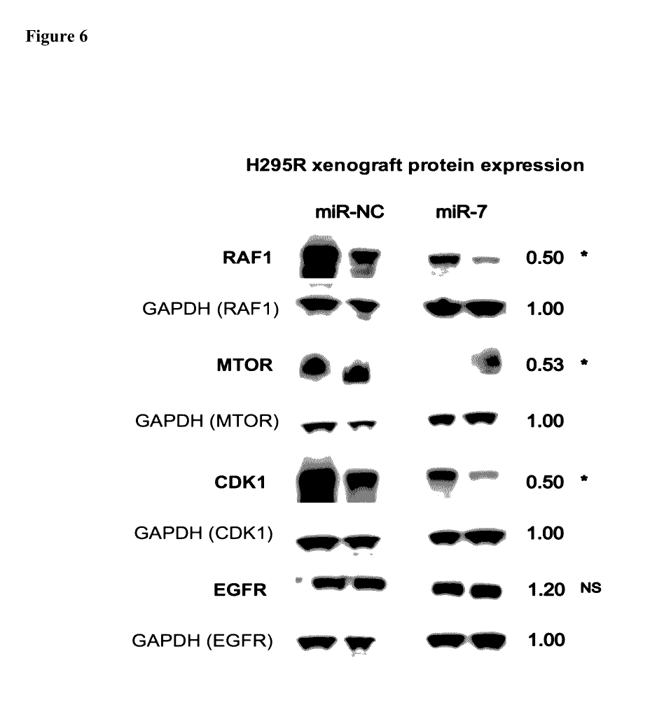

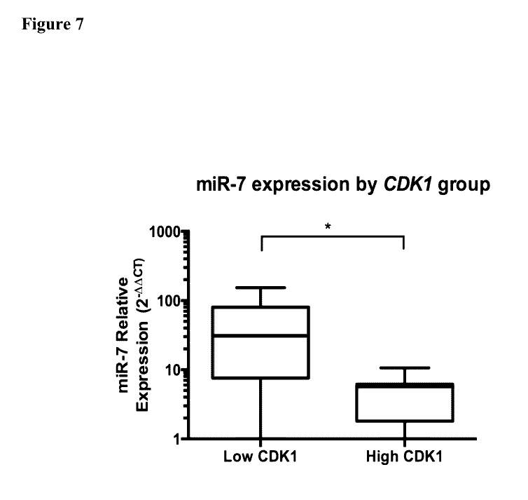

To assess how miR-7 replacement reduces ACC xenograft growth, delivery of miR-7 to the tumor cells by the EDVs was confirmed. RT-qPCR was performed on excised xenografts and showed significantly increased miR-7 expression in miR-7 treated xenografts compared to those treated with miR-NC following six doses of EDVs (FIG. 5A). Further to this, in the miR-7 treated xenografts both RAF1 and MTOR were significantly down-regulated by over 2-fold (FIG. 5E), with reduced protein expression of RAF1 and MTOR detected by Western blotting (FIG. 6). However, no reduction of EGFR expression in the xenograft was detected using RT-qPCR, or Western blotting (FIG. 5E, 6). In addition EGFR expression measured by immunohistochemistry also showed no change between miR-7 and miR-NC treated xenografts. Histopathology showed similar tumor morphology between the miR-7 (FIG. 5J) and miR-NC treated groups (data not shown).

In addition, this analysis also detected significant down-regulation of cyclin-dependent kinase 1 (CDK1) in the miR-7 treated xenografts by RT-qPCR and Western blotting (FIG. 5B, 6). CDK1, not being a predicted target of miR-7 was analysed due to the results of an earlier microarray study. In this analysis which was initially used to study long noncoding RNA and mRNA expression in ACC, differential expressed mRNAs between ACC vs. NAC clinical samples was analysed to identify genes that may be active in ACC [12]. In addition, using Gene Set Enrichment Analysis (GSEA) [16] with KEGG pathway focus (Kanehisha Laboratories, Kyoto, Japan) the cell cycle pathway (KEGG Pathway ID: hsa04110) was found to be the highest enriched up-regulated pathway with 18 genes being significantly over-expressed in ACC vs. NAC (Enrichment score 7.65, P-value=5.9×10⁻⁶). Five of these genes, including CDK1, Pituitary Tumour-Transforming 1 (PTTG1), cyclin B2 (CCNB2), cyclin E1 (CCNE1) and S-phase kinase-associated protein 2 (SKP2), were chosen to test whether these active genes in ACC may be inhibited following miR-7 therapy.

### In Vivo Off-Target Effect Assessment

As with any new treatment modality, the possibility of side effects must be considered. While EDV nanocells have been assessed and found to be safe for human use in phase 1 clinical trials when delivering doxorubicin for recurrent glioma [13], the possibility of side effects from miR-7 itself has not been assessed. Mouse liver, lungs and kidneys were examined by histopathology, miR-7 expression and the expression of molecular targets repressed in the xenografts.

During the study period there was no significant difference in mouse weight between groups and no abnormal behavior or signs of toxicity were seen. No significant change of miR-7 was detected in liver, lung and kidney (FIG. 5B, 5C, 5D) in the miR-7 treated mice in contrast to the significantly increased expression of miR-7 in the xenografts (FIG. 5A). For the molecular endpoints reduced in the xenografts (RAF1, MTOR and CDK1), no significant difference could be seen in the liver, lung or kidney of mice treated with miR-7 compared to miR-NC (FIG. 5F, 5G, 5H). H&E staining on each treated lung, liver and kidney, showed no difference between miR-7 and miR-NC treated groups with normal appearing organs for each treatment group (FIG. 5J, 5K, 5L).

In ACC Patient Samples, miR-7 Expression is Inversely Associated with CDK1 Expression

To further investigate a potential functional relationship between miR-7 and CDK1 the expression of these RNAs in an extended group of ACC clinical samples (n=15) using RT-qPCR was analysed Comparing CDK1 and miR-7 expression analysis by scatter plot did not show a significant linear relationship. However, using a sample splitting method dividing CDK1 expression into high and low groups (by median CDK1 expression), the high CDK1 expression group was found to be associated with a significant lower expression of miR-7 (P=0.04, FIG. 8).

### DISCUSSION

miR-7 acts as a tumor suppressor in ACC and miR-7 replacement therapy reduces ACC xenograft growth. Restoration of miR-7 in vitro reduces cell proliferation and induces G₁ cell cycle arrest. miR-7 replacement in vivo inhibits ACC xenograft growth in models derived from both H295R and primary ACC cells. miR-7 achieves this by directly targeting the MAPK (RAF1) and mTOR signalling pathways (MTOR), leading to inhibition of CDK1.

The nanocells used in these experiments target EGFR, which is also a target of miR-7. As such, it may seem counterintuitive to use this delivery system. Specifically, in providing greater amounts of miR-7 to the ACC cells, one would expect to also see subsequent knock-down of EGFR and over time, reduced efficacy of the miR-7 therapy. Surprisingly, while EGFR knockdown in vitro was detected, it was not possible to detect any significant change in EGFR expression in vivo by RT-qPCR, Western blotting or immunohistochemistry. The cause for these different results is not clear, however these results point to a more complex mechanism of action of miR-7 in vivo than expected. One possible explanation could be that miR-7 reduces but does not abolish the expression of its targets, meaning that a considerable reduction in EGFR expression was not achieved in vivo in the context of an established adrenal cancer xenograft with strong expression of EGFR. Nonetheless, the extent of miR-7 action was significant enough to reduce cell proliferation and induce G1 cell cycle arrest, indicating the positive therapeutic benefits of miR-7 therapy.

This study was designed to investigate whether microRNA therapy has any utility for patients with metastatic ACC who have failed conventional treatment. Given this, miR-7 was delivered in a mouse xenograft model after the tumours were well formed.

### Example 2

### Methods for miR-7 Therapy

Patients with a diagnosis of adrenocortical carcinoma (ACC) are treated with miR-7 therapy in two clinical situations: 1) following surgical resection as an adjuvant therapy and 2) for the treatment of metastatic ACC. Patients are selected for miR-7 therapy on the basis of the levels of miR-7 detected in a sample of their ACC tumour. Specifically, patients are selected for therapy if a biopsy of the ACC or the resected ACC tumour is shown to be deficient in miR-7 as assessed by quantification of miR-7 using RT-qPCR. The amount of miR-7 in the patient sample is compared against a reference database in the form of miR-7 expression amounts detected in various non-neoplastic adrenal cortex samples and other ACC clinical samples.

For adjuvant therapy, miR-7 treatment occurs following surgical resection. The protocol for adjuvant therapy includes three treatments per week by IV infusion of miR-7 packaged in nanoparticles at a dose of 0.32 nmole miR-7 per infusion.

Patients at high risk of recurrence of ACC following surgical resection are treated with miR-7 therapy in addition to adjuvant low-dose mitotane as is the standard current treatment [14]. Patients are identified as having a high risk of ACC recurrence if their tumour size is >8 cm, microscopic invasion of blood vessels/tumour capsule is observed or if the Ki-67 index >10% [3]. Treatment with miR-7 therapy and low-dose mitotane continues for two years, with three to six monthly assessment by clinical examination and surveillance imaging.

Patients with metastatic disease receive miR-7 replacement therapy with three treatments per week by IV infusion of miR-7 packaged in nanoparticles.

miR-7 therapy is provided as the sole treatment for metastatic ACC or is administered in combination with high-dose mitotane as is standard current treatment. High dose mitotane aims for a blood level of 14-20 mg/L and levels should be checked following three weeks of treatment and if plasma levels remain low (<7 mg/L), the commencement of cytotoxic combination chemotherapy (etoposide, doxorubicin and cisplatin) is recommended along with miR-7 and mitotane therapy. After three months of treatment, the patients are assessed by clinical examination and restaged with imaging. For patients with progressive disease, combination cytotoxic chemotherapy (etoposide, doxorubicin and cisplatin) is commenced along with miR-7 therapy.4

Treatment continues for two years or until complete regression of disease, where the patients are recommended for treatment as per the adjuvant therapy protocol.

### SEQUENCES

### SEQ ID NO: 1

miR-7 seed sequence:

### 5′-GGA AGA-3′

### SEQ ID NO: 2

miR-7-5p (mature miR-7 sequence):

### 5′-UGG AAG ACU AGU GAU UUU GUU GU-3′

### REFERENCES

1. Abiven G, Coste J, Groussin L, Anract P, Tissier F, Legmann P, Dousset B, Bertagna X, Bertherat J. Clinical and Biological Features in the Prognosis of Adrenocortical Cancer: Poor Outcome of Cortisol-Secreting Tumors in a Series of 202 Consecutive Patients. Journal of Clinical Endocrinology & Metabolism. 2006 Apr. 18; 91(7):2650-5.
2. Fassnacht M, Terzolo M, Allolio B, Baudin E, Haak H, Berruti A, Welin S, Schade-Brittinger C, Lacroix A, Jarzab B, Sorbye H, Torpy D J, Stepan V, Schteingart D E, et al. Combination Chemotherapy in Advanced Adrenocortical Carcinoma. The New England Journal of Medicine. 2012 Jun. 7; 366(23):2189-97.
3. Scholzen T, Gerdes J. The Ki-67 protein: from the known and the unknown. J Cell Physiol. 2000 March; 182(3):311-22.
4. Soon P S H, Tacon L J, Gill A J, Bambach C P, Sywak M S, Campbell P R, Yeh M W, Wong S G, Clifton-Bligh R J, Robinson B G, Sidhu S B miR-195 and miR-483-5p Identified as Predictors of Poor Prognosis in Adrenocortical Cancer. Clinical Cancer Research. 2009 Dec. 14; 15(24):7684-92.
5. Pasquinelli A E. MicroRNAs and their targets: recognition, regulation and an emerging reciprocal relationship. Nature Reviews Genetics; 2012 April; 13(4):271-82.
6. Cloonan N. Re-thinking miRNA-mRNA interactions: Intertwining issues confound target discovery. BioEssays. 2015 Feb. 12; 37(4):379-88.
7. Wooten M D, King D K. Adrenal cortical carcinoma. Epidemiology and treatment with mitotane and a review of the literature. Cancer. 1993 Dec. 1; 72(11):3145-55.
8. Fassnacht, M. & Allolio, B. What is the best approach to an apparently nonmetastatic adrenocortical carcinoma? Clinical Endocrinology 73, 561-565 (2010).
9. Lau S. K and Weiss L M. Human Pathology, 2009 June 40(6):757-68.
10, MacDiarmid J A, Amaro-Mugridge N B, Madrid-Weiss J, Sedliarou I, Wetzel S, Kochar K, Brahmbhatt V N, Phillips L, Pattison S T, Petti C, Stillman B, Graham R M, Brahmbhatt H. Sequential treatment of drug-resistant tumors with targeted minicells containing siRNA or a cytotoxic drug. Nat Biotechnol. 2009 Jun. 28; 27(7):643-51.
11. Wang T, Rainey W E. Human adrenocortical carcinoma cell lines. Molecular and Cellular Endocrinology; 2012 Mar. 31; 351(1):58-65.
12. Glover A R, Zhao J T, Ip J C, Lee J C, Robinson B G, Gill A J, Soon P S H, Sidhu S B. Long noncoding RNA profiles of adrenocortical cancer can be used to predict recurrence. Endocrine-Related Cancer. 2015 February; 22(1):99-109.
13. MacDiarmid J A, Brahmbhatt H. Targeted EDV™ Nanocells as Versatile Vectors for Delivery of Therapeutics in Cancer: Translating a Platform Technology into Human Clinical Trials. Asia-Pac J Clin Oncol. 2014 Dec. 7; 10(Supp 7):14.
14. Kirschner. L S. The next generation of therapies for adrenocortical cancers. Trends in Endocrinology & Metabolism. Elsevier Ltd; 2012 Jul. 1; 23(7):343-50.

1. A method of treating adrenocortical carcinoma (ACC) in an individual, the method including providing a therapeutically effective amount of a miR-7 microRNA in the individual, thereby treating ACC in the individual.

2. The method according to claim 1 wherein the miR-7 microRNA is provided in an ACC cell of the individual.

3. The method according to claim 2 wherein the amount of miR-7 microRNA provided in the ACC cell is at least the same as the amount of miR-7 microRNA in a non-cancerous adrenal cortex cell.

4. The method according to claim 1, wherein the miR-7 microRNA includes:
a) a first oligonucleotide of between 17-25 nucleotides having a sequence that is 80-100% identical to the nucleotide sequence of SEQ ID NO:2
b) a second oligonucleotide of between 17-25 nucleotides having a sequence that is 60-100% identical to the sequence complementary to the nucleotide sequence of SEQ ID NO: 2.

5. The method according to claim 1 wherein the miR-7 microRNA includes a chemical modification.

6. The method according to claim 1, wherein miR-7 microRNA is provided in the individual by administering a nucleic acid construct to the individual for formation of miR-7 microRNA in the individual.

7. The method according to claim 6 wherein the nucleic acid construct includes:
a coding region for encoding a double stranded RNA molecule;
a promoter operable in a cell for the production of a double stranded RNA molecule;
wherein the double stranded RNA molecule has a sequence that enables the production of a miR-7 microRNA.

8. The method according to claim 1 wherein the method further includes simultaneous or co-administration of a further anti-cancer therapy.

9. An anti-cancer agent in the form of a nanoparticle containing a nucleic acid construct, wherein the nucleic acid construct includes:
a coding region for encoding a double stranded RNA molecule;
a promoter operable in a cell for the production of a double stranded RNA molecule;
wherein the double stranded RNA molecule has a sequence that enables the production of a miR-7 microRNA.

10. An anti-cancer agent in the form of a nanoparticle having an miR-7 microRNA attached thereto.

11. The anti-cancer agent according to claim 10, wherein the miR-7 microRNA is contained within the nanoparticle.

12. The agent according to claim 11, wherein the nanoparticle is derived from a bacterium.

13. The anti-cancer agent according to claim 12, wherein the miR-7 microRNA includes a nucleotide sequence that is at least 80% identical to the nucleotide sequence of SEQ ID NO: 2.

14. The anti-cancer agent according to claim 13 wherein the miR-7 microRNA includes:
a) a first oligonucleotide of between 17-25 nucleotides having a sequence that is between 80% to 100% identical to the nucleotide sequence of SEQ ID NO:2;
b) a second oligonucleotide of between 17-25 nucleotides having a sequence that is between 60% to 100% identical to the nucleic acid sequence complementary to the nucleotide sequence of SEQ ID NO: 2.

15. The anti-cancer agent according to claim 14, wherein the miR-7 microRNA further comprises a chemical modification.

16. A pharmaceutical composition including the agent of claim 15.

17. The agent according to claim 10, wherein the nanoparticle comprises an antibody for targeting the miR-7 microRNA to a cell or tissue to which the miR-7 microRNA is to be provided.

18. Use of an anti-cancer agent according to claim 10 in the manufacture of a medicament for treating cancer in an individual.

19. The use according to claim 18 wherein the cancer is an adrenocortical carcinoma.
